(19) United States
(12) Patent Application Publication
(10) Pub. No.: US 2025/0325938 A1
(43) Pub. Date: Oct. 23, 2025

(54) PLANT AND METHOD FOR PRODUCING BIOMETHANE

(71) Applicant: L'Air Liquide, Société Anonyme pour I'Etude et I'Exploitation des Procédés Georges Claude, Paris (FR)

(72) Inventors: Solène VALENTIN, Voreppe (FR); Francois BARRAUD, Shanghai (CN); Daniel GARY, Montingy le Bretonneux (FR)

(21) Appl. No.: 18/867,659

(22) PCT Filed: May 10, 2023
(86) PCT No.: PCT/EP2023/062466

§ 371 Date: Nov. 20, 2024

(30) Foreign Application Priority Data
May 20, 2022 (FR) FR2204864

(51) Int. Cl. B01D 53/22; B01D 53/04; B01D 53/14; B01D 53/86

(52) U.S. Cl. CPC B01D 53/226; B01D 53/04; B01D 53/14; B01D 53/8671; B01D 2256/245; B01D 2257/104; B01D 2257/504; B01D 2258/05; B01D 2311/10; B01D 2311/14

(57) ABSTRACT

The invention relates to a plant and method for producing deoxygenated biomethane (10) having an oxygen concentration below a determined threshold, in particular below 100 ppm, from biogas (1), the plant comprising:—a membrane-permeation treatment unit (5) which is able to produce biomethane (9) and is configured to produce biomethane having a molar concentration below a first determined threshold, for example less than 5% CO₂ and less than 1% O₂, in particular less than 3% CO₂ and less than 0.7% O₂, from a stream of biogas having a CO₂ concentration above a second threshold, for example from 15 to 60% carbon dioxide,—a compressor (2) configured to compress the biogas (1), and—at least one catalytic reaction unit (3) comprising at least one bed of at least one oxidation catalyst configured to deoxygenate the biogas (1) and/or the biomethane (9) and/or the partially purified biogas (7).

### CROSS REFERENCE TO RELATED APPLICATIONS

This application is a 371 of International Application No. PCT/EP2023/062466, filed May 10, 2023, which claims priority to French Patent Application No. 2204864, filed May 20, 2022, the entire contents of which are incorporated herein by reference.

### BACKGROUND

The present invention relates to an installation and process for the production of biomethane.

The invention relates more particularly to an installation for the production of deoxygenated biomethane having an oxygen concentration below a predetermined threshold, in particular below 100 ppm, starting from biogas, and also to a corresponding process.

Biogas is the gas produced during the decomposition of organic matter in the absence of oxygen (anaerobic fermentation), also known as methanization. This can be a natural decomposition which is observed in marshes or household waste landfills. Biogas production can also result from the methanization of waste in a dedicated reactor, the conditions of which are controlled, called a methanizer or digester, then in a post-digester, similar to the digester and making it possible to push the methanization reaction further.

Biomass refers to any group of organic matter which can be transformed into energy through this methanization process, for example treatment plant sludge, manure/slurry, agricultural residues and food waste.

Biogas contains predominantly methane (CH₄) and carbon dioxide (CO₂) in proportions which can vary depending on the mode of acquisition and on the substrate but can also contain, in lesser proportions, water, nitrogen, hydrogen sulfide (H₂S), oxygen, and also other organic compounds, in the form of traces, including H₂S, between 10 and 50 000 ppmv.

According to the organic matter decomposed and the techniques used, the proportions of the components differ but, on average, biogas comprises (in moles or by volume), on a dry gas basis, from 30% to 75% of methane, from 15% to 60% of carbon dioxide, from 0% to 15% of nitrogen, from 0% to 5% of oxygen, and compounds in the form of traces, such as sulfur compounds, chlorine compounds, halogen compounds and volatile organic compounds (VOCs).

Biogas can be upgraded in various ways. It can, after a mild treatment, be upgraded close to the production site to provide heat, electricity or a mixture of the two (cogeneration); the high content of carbon dioxide reduces its calorific value, increases the costs of compression and of transportation, and limits the economic advantage of its upgrading to this nearby use.

A more exhaustive purification of the biogas makes possible its wider use; in particular, an exhaustive purification of the biogas makes it possible to obtain a biogas which is purified to the specifications of natural gas and which can be substituted for it. The biogas thus purified is referred to as “biomethane”. Biomethane thus supplements natural gas resources with a renewable part produced within territories; it can be used for exactly the same uses as natural gas of fossil origin. It can feed a natural gas network or a vehicle filling station; it can also be liquefied to be stored in the form of liquefied natural gas (bioLNG).

In the context of the development of a solution for clean transport, it is a matter of synthesizing biomethane in order to provide a renewable fuel to “VNG” or “LNG” vehicles. Some natural gas networks impose a limitation on the oxygen content (for example, 100 ppm maximum).

Deoxygenation systems exist. The catalytic deoxygenation of argon is usually carried out at low temperature, for example below 200° C., and the deoxygenation of methane or of biogas by catalysis is carried out at a higher temperature, for example above 200° C.

The document U.S. Pat. No. 11,219,889 B2 describes a process for the preparation of a methane oxidation catalyst based on precious metal impregnated on ZrO₂ for deoxygenating an exhaust gas from a natural gas engine.

A document published in the context of the International Gas Union conference (IGRC2014) describes the removal of oxygen from biogas by catalytic oxidation of methane, in which methane is used as agent which reduces the oxygen.

The known solutions are not suitable for the production of biomethane.

Conventionally, the biogas passes through a bed of activated carbon which removes impurities referred to as poisons, such as sulfur compounds (H₂S, C₂H₆S, COS, and the like). If these carbons are not replaced in time, or if they are failing, these impurities can become poisons of a system for deoxygenation by the catalytic route.

The disadvantage of the existing systems is that ultrapurification of the biogas is not guaranteed or stable over time.

### SUMMARY

To this end, the installation according to the invention, which is otherwise in accordance with the generic definition which is given of it in the preamble above, is essentially characterized in that it comprises:
a unit for the purification of the biogas capable of producing biomethane, configured in order to produce biomethane having a molar or volume concentration below a first predetermined threshold, for example less than 5% of CO₂ and less than 1% of O₂, in particular less than 3% of CO₂ and less than 0.7% of O₂, starting from a biogas stream having a concentration of CO₂ above a second threshold, for example from 15% to 60% of carbon dioxide,
a compressor configured to compress the biogas, and
at least one catalytic reaction unit (or one catalytic reactor) comprising at least one bed of at least one oxidation catalyst configured to deoxygenate the biogas and/or the biomethane and/or the partially purified biogas.

This invention provides for a catalytic reactor in the procedure for purification of the biogas in order to very significantly remove the oxygen present in the biomethane.

Such an installation makes it possible to achieve ultrapurification of the biogas, the O₂ content present in the biomethane being reduced to a level below 100 ppm, in particular below 1 ppm.

Moreover, embodiments of the invention can comprise one or more of the following characteristics:
a device for control of the pressure within the purification unit and/or within the catalytic reaction unit,
a unit for control of the operating temperature within the catalytic reaction unit,
an electronic controller comprising a microprocessor, said controller being configured to regulate the composition of the deoxygenated biomethane produced by operating the device for control of the pressure within the purification unit and/or within the catalytic reaction unit, and/or by operating the unit for control of the operating temperature within the catalytic reaction unit,
a pretreatment unit comprising a booster, a means for drying by condensation of the water and at least one bed of activated carbons which is configured to pretreat the biogas,
at least one impurity removal unit (the impurities being, for example, sulfur compounds, chlorine compounds, halogen compounds and VOCs) comprising at least one guard bed, the impurity removal unit being located upstream of the at least one catalytic reaction unit, said guard bed comprising particles of at least one metal oxide of at least one metal chosen from transition metals, said guard bed being placed upstream of said at least one bed of at least one oxidation catalyst,
the unit for the purification of the biogas comprises a unit of PSA type and/or a unit of scrubbing type and/or a unit for treatment by membrane permeation comprising at least two membrane separation units in parallel and/or in series, for example three membrane separation units (or stages), two units of which are placed in series and one unit of which is placed in parallel with one of the units in series, each membrane separation unit comprising one or more membranes connected in parallel,
the purification unit is or comprises a unit for treatment by membrane permeation comprising: a first membrane separation unit equipped with a first membrane able and configured to receive the biogas and to provide a first permeate and a first retentate, said first membrane being more permeable to carbon dioxide than to methane, a second membrane separation unit equipped with a second membrane able and configured to receive the first retentate and to provide a second permeate and a second retentate, said second membrane being more permeable to carbon dioxide than to methane and said second retentate being the biomethane, and a third membrane separation unit equipped with a third membrane able and configured to receive the first permeate and to provide a third permeate and a third retentate, said third membrane being more permeable to carbon dioxide than to methane,
the device for control of the pressure comprises a pressure-control valve located in or downstream of the unit for purification of the biogas or of the second membrane separation unit,
the catalytic reaction unit is arranged upstream of the unit for purification of the biogas or of the first membrane separation unit, the catalytic reaction unit being configured to bring the biogas into contact with at least one bed of at least one oxidation catalyst of the catalytic reaction unit,
the catalytic reaction unit is arranged downstream of the unit for purification of the biogas or of the second membrane separation unit and configured to bring the biomethane obtained resulting from the unit for purification of the biogas into contact with at least one bed of at least one oxidation catalyst of the catalytic reaction unit,
the catalytic reaction unit is arranged downstream of the first membrane separation unit and configured to bring the first retentate into contact with at least one bed of at least one oxidation catalyst of the catalytic reaction unit,
the installation comprises at least one heat exchanger, configured to heat the at least one catalytic reaction unit and/or the gas entering the catalytic reaction unit.

The invention also relates to a process for the production of deoxygenated biomethane having an oxygen concentration below a predetermined threshold, in particular below 100 ppm, starting from biogas, the process comprising the following steps:
a step of making available biogas containing, in molar or volume proportion, from 30% to 75% of methane, from 15% to 60% of carbon dioxide, and also one at least from: water, nitrogen, hydrogen sulfide, oxygen and/or volatile organic compounds (VOCs),
a step of compression of the biogas in a compressor,
a step of purification of the biogas in a unit for separation of carbon dioxide from methane to produce biomethane, said biomethane comprising, in molar or in volume proportion, less than 3% of CO₂ and less than 0.7% of O₂, and
a deoxygenation step in which the biogas and/or the biomethane and/or the partially purified biogas is deoxygenated in at least one catalytic reaction unit comprising at least one bed of at least one oxidation catalyst, in particular a methane oxidation catalyst, for respectively producing deoxygenated biogas and/or deoxygenated biomethane and/or deoxygenated partially purified biogas.

According to other possible distinguishing features:
the process comprises a step of control of the quality of the deoxygenated biomethane, in particular its carbon dioxide composition, by regulating the pressure within the purification unit and the catalytic reaction unit,
the process comprises a step of control of the quality of the deoxygenated biomethane, in particular its oxygen composition, by regulating the operating temperature within the catalytic reaction unit,
the process comprises a step of pretreatment of the biogas, in particular before the compression step, the pretreatment step being configured to remove at least a part of the water and/or of the hydrogen sulfide and/or of the VOCs present in the biogas,
the process comprises, before the deoxygenation step, a step of removal of at least one impurity selected from sulfur compounds, chlorine compounds, halogen compounds and VOCs, by bringing the biogas and/or the biomethane and/or the partially purified biogas into contact with at least one impurity removal unit, the impurity removal unit comprising at least one guard bed comprising particles of at least one metal oxide of at least one metal chosen from transition metals,
the step of purification of the biogas comprises a treatment by membrane separation and/or a treatment by adsorption by means of a unit of PSA type and/or a treatment by absorption by means of a scrubbing column,
the step of purification of the biogas comprises a treatment by membrane separation comprising at least two membrane separation units in parallel and/or in series, for example three membrane separation units (or stages), two units of which are placed in series and one unit of which is placed in parallel with one of the two units in series, each membrane separation unit comprising one or more membranes connected in parallel,
the step of purification of the biogas comprises a treatment by membrane separation comprising at least the following steps: a step (a) of bringing the biogas into contact with a first membrane separation unit so as to produce a first permeate enriched in carbon dioxide with respect to the biogas and a first retentate enriched in methane with respect to the biogas, a step (b) of bringing the first retentate into contact with a second membrane separation unit so as to produce a second permeate enriched in carbon dioxide with respect to the first retentate and a second retentate enriched in methane with respect to the first retentate, the second retentate being the biomethane, and a step (c) of bringing the first permeate into contact with a third membrane separation unit so as to produce a third permeate enriched in carbon dioxide with respect to the first permeate and a third retentate enriched in methane with respect to the first permeate,
the second permeate and/or the third retentate is recycled upstream of the compressor,
the deoxygenation step is carried out before the step of purification of the biogas by bringing the biogas into contact with at least one bed of at least one oxidation catalyst of the catalytic reaction unit,
the deoxygenation step is carried out by bringing the biomethane obtained after the step of purification of the biogas into contact with at least one bed of at least one oxidation catalyst of the catalytic reaction unit,
the deoxygenation step is carried out by bringing the first retentate into contact with at least one bed of at least one oxidation catalyst of the catalytic reaction unit,
the at least one bed of at least one oxidation catalyst comprises the particles of at least one precious metal, for example chosen from: Pd, Pt and Rh, deposited on at least one inorganic metal oxide, for example chosen from: Al₂O₃, ZrO₂ and TiO₂, or the particles of at least one transition metal, for example chosen from: Cu and Ni, deposited on or mixed at least with an inorganic metal oxide, for example chosen from: ZnO, MgO and CaO,
the deoxygenation step is carried out at a temperature of between 280° C. and 450° C., preferably between 280° C. and 390° C.,
the deoxygenation step is carried out at a pressure greater than atmospheric pressure, preferably between 5 and 20 bar, in particular between 8 and 15 bar.

The term “biogas” is understood to mean the crude biogas or the crude biogas stream exiting from a biogas production unit, in particular a digester, containing, in moles or by volume, from 30% to 75% of methane, from 15% to 60% of carbon dioxide, and also one at least from: water, nitrogen, hydrogen sulfide, oxygen and/or volatile organic compounds (VOCs).

The term “deoxygenated biogas” is understood to mean without distinction the biogas or the biogas stream exiting from the catalytic reaction unit after the deoxygenation step.

The term “biomethane” is understood to mean without distinction the biomethane and the biomethane stream exiting from the unit for purification of the biogas, or the purified biogas, comprising, by molar mass or by volume, for example less than 5% of carbon dioxide and less than 1% of oxygen (volume and molar amount being equivalent in the case where the ideal gas equation is used).

The term “deoxygenated biomethane” is understood to mean without distinction the biomethane and the biomethane stream exiting from the catalytic reaction unit after the deoxygenation step.

The term “partially purified biogas” is understood to mean the biogas in the course of purification to remove or reduce the content of impurities and/or of carbon dioxide or the biogas withdrawn from the purification unit, enriched in methane with respect to the biogas and enriched in carbon dioxide with respect to the biomethane. A partially purified biogas is, for example, a first retentate exiting from the first membrane separation unit.

The term “deoxygenated partially purified biogas” is understood to mean without distinction the partially purified biogas and the partially purified biogas stream or a first retentate exiting from the catalytic reaction unit after the deoxygenation step.

The term “guard bed” is understood to mean a bed of protective particles targeted at trapping impurities known as poisons, such as sulfur compounds, chlorine compounds, halogen compounds or VOCs, liable to be contained in the biogas or the biomethane or the partially purified biogas before entering the catalytic reaction unit.

The invention can also concern any alternative device or process comprising any combination of the characteristics given above or below.

### BRIEF DESCRIPTION OF THE FIGURES

Other distinguishing features and advantages will become apparent on reading the description below, made with reference to the figures, in which:

FIG. 1 represents a schematic and partial view illustrating a first implementational example of structure and of operation of an installation according to the invention,

FIG. 2 represents a schematic and partial view illustrating a second implementational example of structure and of operation of an installation according to the invention,

FIG. 3 represents a schematic and partial view illustrating a third implementational example of structure and of operation of an installation according to the invention,

FIG. 4 represents a schematic and partial view illustrating a fourth implementational example of structure and of operation of an installation according to the invention,

FIG. 5 represents a schematic and partial view illustrating a fifth implementational example of structure and of operation of an installation according to the invention,

FIG. 6 represents a schematic and partial view illustrating a sixth implementational example of structure and of operation of an installation according to the invention,

FIG. 7 represents a schematic and partial view illustrating an implementational example of structure and of operation of a catalytic reactor which can be used within the installation according to the invention.

### DETAILED DESCRIPTION OF PREFERRED EMBODIMENTS

Throughout the figures, the same references relate to the same elements.

In this detailed description, the following implementations are examples. Although the description refers to one or more embodiments, this does not mean that the characteristics apply only to a single embodiment. Single characteristics of different embodiments can also be combined and/or interchanged in order to provide other implementations.

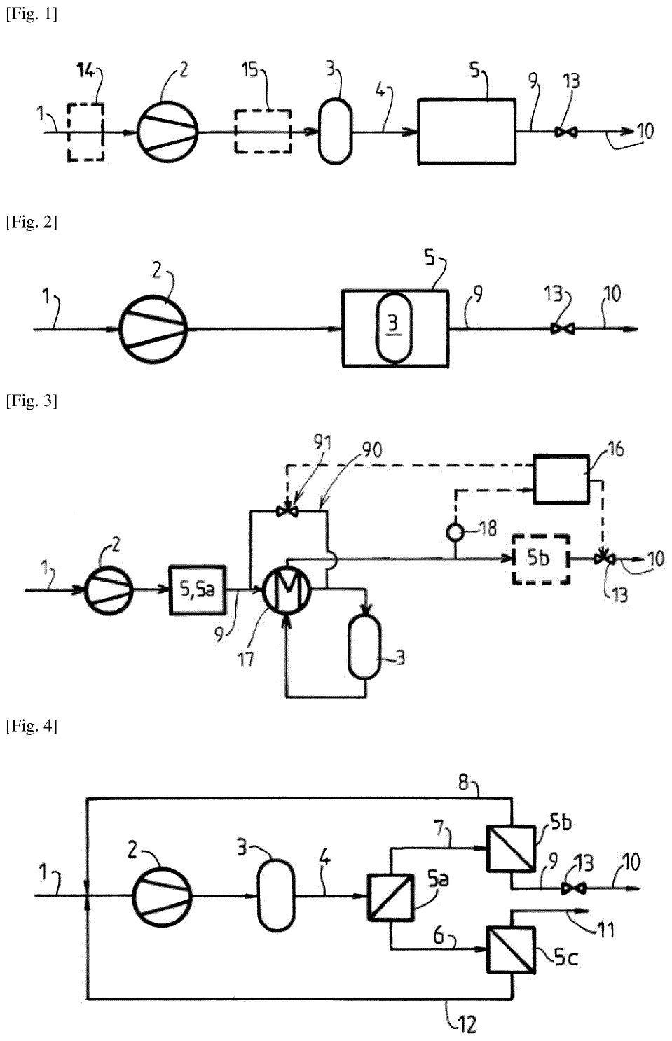

The installation represented in FIG. 1 is an example of a device for the production of biomethane 10. The installation comprises a fluid circuit comprising an upstream end intended to be connected to a biogas source, for example the outlet of a biogas production unit, in particular a digester, to receive a biogas stream 1 and a downstream end configured to provide the biomethane 10.

The installation comprises, between its upstream and downstream ends, a biogas purification unit 5, in particular a unit for the separation of carbon dioxide, able and configured to produce biomethane 9, the essential (predominant) component of which is methane (CH₄) and additionally comprising, in moles or by volume, less than 5% of CO₂ and less than 1% of O₂, in particular less than 3% of CO₂ and less than 0.7% of O₂. The installation can comprise, upstream of the purification unit 5, a heat exchanger for adjusting the temperature of the feed gas stream of the purification unit 5.

The purification unit 5 can comprise a unit of PSA type and/or a unit of scrubbing type, in particular a unit for treatment by absorption by means of a scrubbing column and/or a unit for treatment by membrane permeation comprising, for example, at least two membrane separation units.

In the case having two membrane separation units (or stages), these units are, for example, connected in series and/or in parallel in the circuit. Of course, the unit for treatment by membrane permeation can comprise three or four membrane separation units (or more). Each membrane separation unit can comprise one or more membranes connected in parallel. Preferably, the unit for treatment by membrane permeation comprises three membrane separation units, the first membrane separation unit and the second membrane separation unit of which are arranged in series in the circuit. The first membrane separation unit and the third membrane separation unit are for their part connected in parallel. One of the streams resulting from the second membrane separation unit and/or one of the streams resulting from the third membrane separation unit can be recycled to the feeding of the unit for treatment by membrane permeation.

To achieve the quality of the biomethane required in terms of level of carbon dioxide within the unit for treatment by membrane permeation, the installation can comprise at least one pressure control device, such as one or more proportional valves, and/or at least one unit for control of the temperature, such as one or more heat exchangers.

The installation comprises at least one catalytic reaction unit 3 comprising at least one bed of at least one oxidation catalyst configured to deoxygenate the biogas before it enters the purification unit 5. That is to say that the catalytic reaction unit 3 comprising at least one bed of at least one oxidation catalyst is located upstream of the purification unit 5 in order to deoxygenate the biogas stream 1. The catalytic reaction unit 3 is configured to bring the biogas into contact with at least one bed of at least one oxidation catalyst of the catalytic reaction unit 3. In particular, the oxidation catalyst is preferably a catalyst of the oxidation of methane.

This oxidation catalyst comprises a catalyst bed which can comprise particles of at least one precious metal, for example chosen from Pd, Pt and Rh. For example, one or more of these precious metals is deposited on at least one inorganic metal oxide, for example chosen from Al₂O₃, ZrO₂, TiO₂, ZnO, MgO and CaO, in particular chosen from Al₂O₃, ZrO₂ and TiO₂. The bed of the oxidation catalyst (in particular a catalyst of the oxidation of methane) can comprise particles of at least one transition metal, for example chosen from Cu and Ni. One or more of these metals is deposited on or mixed at least with an inorganic metal oxide, for example chosen from Al₂O₃, ZrO₂, TiO₂, ZnO, MgO and CaO, in particular chosen from ZnO, MgO and CaO.

In this example, the installation additionally comprises a compressor 2 upstream of the catalytic reaction unit 3. Of course, this compressor might be arranged downstream of the catalytic reaction unit 3.

The compressor 2 is a device or system configured to compress the crude biogas 1 received from a biogas production unit upstream of the installation and/or the pretreated biogas. The compressor 2 can be a medium-pressure compressor lubricated with oil or with water or not, configured to increase the pressure and to make it possible to efficiently provide the separation of the carbon dioxide from the methane in the purification unit 5. An oil removal system can be placed downstream of the compressor 2 to prevent contamination of the purification unit 5 and/or the catalytic reaction unit 3 by the oil.

Advantageously, the installation can comprise, in particular upstream of the compressor 2 and of the catalytic reaction unit 3, a pretreatment unit 14 configured to remove at least a part of the water and/or of the hydrogen sulfide and/or the VOCs present in the biogas 1.

This pretreatment unit 14 can comprise a booster, a blower or a compressor to have sufficient pressure for the passage of the gas through the other steps and/or a unit for drying by condensation of the water, in particular at 5° C., and/or at least one bed of activated carbons to preferentially remove the hydrogen sulfide and the VOCs.

The pretreatment unit 14 can be laid out starting with a drying unit configured to remove at least a part of the water included in the crude biogas 1. The crude biogas exiting from the drying unit can be subsequently received by a blower to reach the pressure sufficient for the gas to pass through the following steps. Downstream of the blower, the installation can comprise at least one hydrogen sulfide removal unit, in particular two hydrogen sulfide removal units, connected in parallel, the activated carbon of which is chosen to preferentially remove the hydrogen sulfide present in the biogas 1. The installation can also comprise a unit for removal of the VOCs which is connected in series with regard to the hydrogen sulfide removal unit and the activated carbon of which is chosen to preferentially remove the VOCs present in the biogas 1.

In an alternative form or in combination, the installation can comprise, upstream of the catalytic reaction unit 3 and/or upstream of the purification unit 5, an impurity removal unit 15 configured to remove at least one impurity chosen from sulfur compounds, chlorine compounds, halogen compounds and VOCs.

The unit 15 for removal of impurities can comprise at least one guard bed comprising particles of at least one metal oxide of at least one metal chosen from transition metals. In particular, the at least one guard bed comprises particles of at least one metal oxide, for example a metal peroxide, a transition metal oxide and/or a transition metal oxide doped with another transition metal.

By way of example, the at least one guard bed comprises a mixture comprising at least one, two or several or all of the following oxides: a zinc oxide, a copper-doped zinc oxide, an alumina, a potassium-doped alumina and a manganese peroxide.

The removal of impurities in the unit 15 for removal of impurities is, for example, carried out at a temperature of greater than or equal to 150° C., in particular between 150° C. and 400° C., preferably between 300° C. and 400° C. A heat exchanger or heater can be provided to this end, for example upstream of or in said unit 15 for removal of impurities.

In a particular embodiment, the impurity removal unit 15 and the catalytic reaction unit 3 can be integrated into a single catalytic reactor. More particularly, the at least one guard bed and the at least one bed of at least one oxidation catalyst can be integrated into a single catalytic reactor. The feed gas of the reactor thus passes through the at least one guard bed and subsequently through the at least one oxidation catalyst.

The installation also preferably comprises a pressure-control valve 13 located in or downstream of the unit 5 for purification of the biogas. The pressure-control valve 13 is, for example, a valve of the proportional type and configured to control the pressure of the feeding of the purification unit 5. The opening/closing of this valve 13 makes it possible to adjust the pressure within the purification unit 5 and/or within the catalytic reaction unit 3. The feed pressure of the purification unit 5 makes it possible to ensure the quality of the biomethane in terms of carbon dioxide. The proportional valve 13 is closed (at least partially) and thus the feed pressure of the purification unit 5 is increased, when the carbon dioxide level is greater than the required quality. The proportional valve 13 is open (at least partially) and thus the feed pressure of the purification unit 5 is reduced, when the carbon dioxide level is lower than the required quality.

By placing the catalytic reaction unit 3 for the removal of the oxygen upstream of the purification unit 5, carbon dioxide is produced in the catalytic reaction unit 3 by the catalytic reaction for oxidation of the methane. The carbon dioxide produced can thus be removed by the purification unit 5 to achieve the required quality of carbon dioxide in the biomethane 10.

The installation represented in FIG. 2 is another example of a device for the production of biomethane 10. The embodiment of FIG. 2 differs from that of FIG. 1 essentially in that the catalytic reaction unit 3 is integrated into the unit 5 for purification of the biogas, for example between different membrane separation units or stages when the purification unit is a unit for treatment by membrane permeation.

The installation represented in FIG. 3 is another example of a device for the production of biomethane 10 which differs from the preceding examples in that the catalytic reaction unit 3 is located downstream of the purification unit 5. In this configuration, the catalytic reaction unit 3 is configured to bring the biomethane obtained resulting from the purification unit 5 into contact with at least one bed of at least one oxidation catalyst of the catalytic reaction unit 3. The biomethane brought into contact with at least one bed of at least one oxidation catalyst of the catalytic reaction unit 3 comprises, for example, in moles or by volume, less than 5% of CO₂ and less than 1% of O₂, in particular less than 3% of CO₂ and less than 0.7% of O₂.

As above, the installation can comprise a compressor 2 located upstream of the catalytic reaction unit 3 and/or upstream of the purification unit 5 and/or downstream of the purification unit 5. The compressor 2 can be configured to compress the crude biogas 1 received directly from a biogas production unit upstream of the installation and/or the biomethane exiting from the purification unit 5.

As described in greater detail below, the installation can also comprise at least one heat exchanger or heater 17 configured to heat the at least one catalytic reaction unit 3 and/or the gas entering the catalytic reaction unit 3. The at least one heat exchanger or heater 17 can be located upstream of the catalytic reaction unit 3 and configured to heat the feed gas of the catalytic reaction unit 3, for example by indirect contact with the gas produced by the catalytic reaction unit 3.

As shown schematically in FIG. 3, the installation can comprise a unit for control of the temperature of the gas, in particular produced by the catalytic reaction unit 3, in order to ensure the quality of the gas at the outlet of the catalytic reactor, in terms of oxygen content. Such a unit for control of the temperature can make it possible to ensure the quality of the deoxygenated biomethane produced, in terms of oxygen content in the biomethane. The unit for control of the temperature comprises, for example, a heat exchanger 17 making possible indirect contact of the feed gas and of the product gas from the catalytic reaction unit 3. The unit for control of the temperature can comprise a bypass line 90 of the heat exchanger 17 including a valve 91, for example a proportional valve, in order to control/limit the flow rate of the feed gas which is circulated in the heat exchanger 17 before entering the catalytic reaction unit 3, and a controller 16, for example a PLC or a computer, comprising a microprocessor. The controller 16 can be configured to control or limit the flow rate of the gas, for example produced by the catalytic reaction unit 3, which circulates in the heat exchanger 17 by opening the proportional valve 91 when the temperature of the product gas is greater than the operating temperature for obtaining the correct oxygen content or the composition of the deoxygenated gas, in particular for obtaining the composition of the deoxygenated biomethane 10. The operating temperature within the catalytic reaction unit 3 is in particular between 280° C. and 450° C., preferably between 280° C. and 390° C. or between 380° C. and 420° C.

To this end, the installation can comprise an analyzer 18, for example an oxygen analyzer, which analyzes the gas stream in the circuit and the measurement signal of which is sent to the controller 16. For example, in the case of detection of an excessively high oxygen content (above a predetermined threshold), the controller 16 proceeds to close the proportional valve 91 in order to obtain the increase in the temperature via the heat exchanger 17. The analyzer 18 is preferably located downstream of the purification unit 5 and of the catalytic reaction unit 3.

The installation also preferably comprises a pressure-control valve 13 located downstream of the unit 5 for purification of the biogas and/or of the catalytic reaction unit 3. The pressure-control valve 13 is, for example, a valve of the proportional type and configured to control the feed gas pressure of the purification unit 5. The opening/closing of this valve 13 makes it possible to adjust the pressure within the purification unit 5 and/or within the catalytic reaction unit 3. The operating pressure within the catalytic reaction unit 3 can be maintained above atmospheric pressure, preferably between 5 and 20 bar, in particular between 8 and 15 bar.

The feed gas pressure of the purification unit 5 makes it possible to ensure the quality of the biomethane in terms of carbon dioxide content. The feed gas pressure of the purification unit 5 is in particular between 8 and 15 barg.

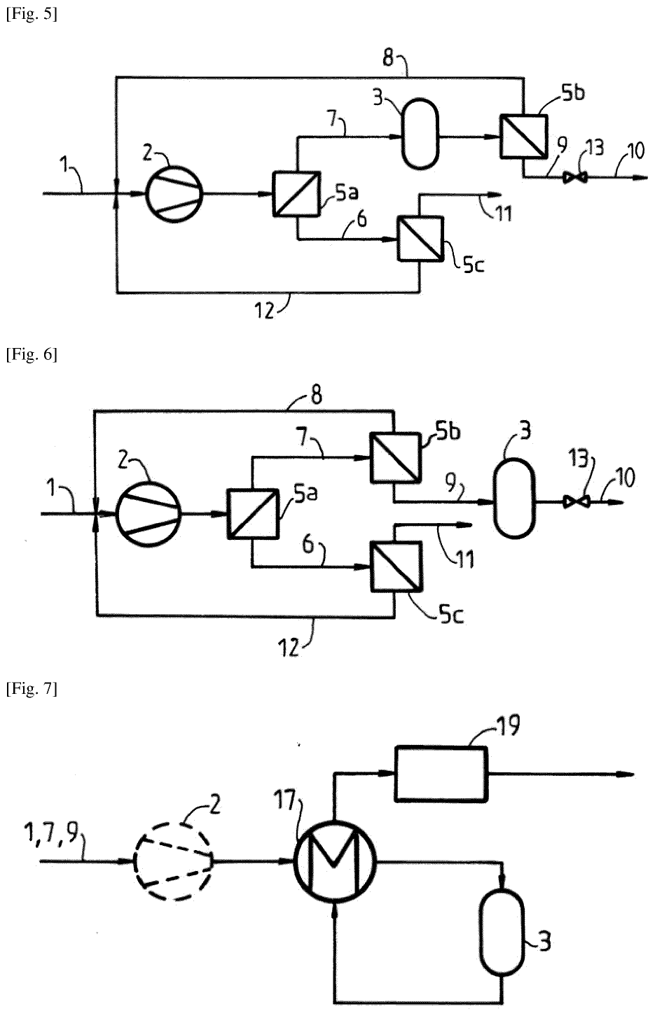

When the level of carbon dioxide is greater than the required quality, for example greater than 3%, the proportional valve 13 is closed and thus the feed pressure of the purification unit 5 is increased. This increases the efficiency of the purification of the carbon dioxide within the purification unit 5. Conversely, when the level of carbon dioxide is less than the required quality, for example less than 2%, the proportional valve 13 is opened, at least partially, and in reaction the feed pressure of the purification unit 5 is reduced. The structure and the operation of the installation which are described above, in particular relating to the control of the quality of the deoxygenated biomethane by the control of the temperature and of the pressure of the catalytic reaction unit 3 and/or of the purification unit 5, can also be applied to the configurations illustrated in FIG. 1, FIG. 2, FIG. 4, FIG. 5, FIG. 6.

By placing the catalytic reaction unit 3 downstream of the purification unit 5, the removal of the carbon dioxide by the purification unit 5 makes it possible to reduce the flow rate treated by the catalytic reaction unit 3.

The installation represented in FIG. 4 is another example of a device for the production of biomethane 10 which differs from the embodiment of FIG. 1 in that the purification unit 5 comprises three membrane separation units. The catalytic reaction unit 3 is in particular located upstream of a first membrane separation unit 5a.

The first membrane separation unit 5a (or the first membrane separation stage) can be equipped with several suitable membranes in parallel and is configured to receive the biogas and to provide a first permeate 6 and a first retentate 7.

The second membrane separation unit 5b (or the second membrane separation stage) can be equipped with several suitable membranes in parallel and is configured to receive the first retentate 7 and to provide a second permeate 8 and a second retentate 9, also referred to as biomethane. The installation can also comprise, upstream of the second membrane separation unit 5b, a heat exchanger (not represented for the sake of simplification) configured to adjust the temperature of the gas stream entering the second membrane separation unit 5b. This heat exchanger makes it possible to ensure the quality of the biomethane 9 in terms of carbon dioxide by increasing the selective permeation of the carbon dioxide by decreasing the temperature, if appropriate.

The third membrane separation unit 5c (or the third membrane separation stage) can be equipped with several membranes in parallel and is able and configured to receive the first permeate 6 and to provide a third permeate 11 and a third retentate 12.

The first membrane separation unit 5a is able to receive the compressed and deoxygenated biogas stream 1 exiting from the catalytic reaction unit 3 and to provide a first permeate 6 enriched in carbon dioxide with respect to the biogas 1 and a first retentate 7 enriched in methane with respect to the biogas 1.

Preferably, the second permeate 8 and/or the third retentate 12 is recycled upstream of the compressor 2.

The pressure-control valve 13 is located downstream of the second membrane separation unit 5b on the second retentate 9, which is the deoxygenated biomethane 10.

The installation can also comprise, downstream of the third retentate 12, a control valve configured to adjust the level of methane in the permeate 11 which is discharged at the vent and thus to limit the loss of methane.

By placing the catalytic reaction unit 3 for the removal of the oxygen upstream of the purification unit 5, carbon dioxide is produced in the catalytic reaction unit 3 by catalytic reaction for oxidation of the methane. The carbon dioxide produced can thus be removed by the purification unit 5 to achieve the required quality of carbon dioxide in the biomethane 10.

The installation represented in FIG. 5 is another example of a device for the production of biomethane 10 which differs from that of FIG. 4 essentially in that catalytic reaction unit 3, comprising at least one bed of at least one oxidation catalyst, is located downstream of the first membrane separation unit 5a and configured to deoxygenate the first retentate 7 before it enters the second membrane separation unit 5b. That is to say that the deoxygenated gas stream, in particular the first deoxygenated retentate 7, exiting from the catalytic reaction unit 3 is subsequently received by the second membrane separation unit 5b to provide the second permeate 8 and the second retentate 9, which is the deoxygenated biomethane 10. The deoxygenation step is carried out in the catalytic reaction unit 3 by bringing the first retentate 7 into contact with at least one bed of at least one oxidation catalyst of the catalytic reaction unit 3.

In this alternative form, the installation can also comprise a unit for control of the temperature of the gas, as illustrated in FIG. 3, in which an analyzer 18 is preferably located downstream of the first membrane separation unit 5a and of the catalytic reaction unit 3. Preferably, a pressure-control valve 13 is located downstream of the second membrane separation unit 5b on the second retentate 9, which is the deoxygenated biomethane 10.

The installation can also comprise, downstream of the catalytic reaction unit 3 and upstream of the second membrane separation unit 5b, a heat exchanger (not represented for the sake of simplification) configured to adjust the temperature of the gas stream entering the second membrane separation unit 5b. Said heat exchanger is preferably located downstream of the unit for control of the temperature of the gas at the outlet of the catalytic reactor 3, such as described above with reference to FIG. 3. This heat exchanger makes it possible to ensure the quality of the biomethane 9 in terms of carbon dioxide by increasing the selective permeation of the carbon dioxide by decreasing the temperature, if appropriate.

By placing the catalytic reaction unit 3 on the retentate 7 from the first membrane separation unit 5a, it is possible to limit the flow rate treated by the catalytic reaction unit 3 and to benefit from the second membrane unit 5b to separate the carbon dioxide produced in the catalytic reaction unit 3. Such a location of the catalytic reaction unit 3 is also advantageous because this device for deoxygenation by catalytic reaction produces water and CO₂. By placing the catalytic reaction unit 3 between the first membrane separation stage 5a and the second membrane separation stage 5b, the second membrane separation stage 5b separates the water and the CO₂ produced in order to achieve the required quality of biomethane. No investment in additional items of equipment is necessary to achieve this quality of biomethane at the outlet of the second membrane separation stage 5b and there is no additional consumption of electricity. In addition, the gas treated by the catalytic oxidation in the catalytic reaction unit 3 still contains a high content of CO₂. This CO₂, which is inert during the reaction, will be heated by exothermicity of the reaction and the maximum temperature reached to obtain the complete conversion will be lower.

The installation represented in FIG. 6 is another example of a device for the production of biomethane 10 which differs from that of FIG. 5 essentially in that the catalytic reaction unit 3 is located downstream of the second membrane separation unit 5b. That is to say that the catalytic reaction unit 3 is able and configured to receive the compressed biomethane stream 9 which is the second retentate 9 (and comprises, by volume, for example, less than 3% of CO₂ and less than 0.7% of O₂, in particular less than 3% of CO₂ and less than 0.7% of O₂).

Placing the catalytic reaction unit 3 on the retentate 9 from the second membrane separation unit 5b makes it possible to reduce the flow rate treated by the catalytic reaction unit.

FIG. 7 represents an implementational example of structure and of operation of a catalytic reaction unit 3, in particular of a catalytic reactor which can be used in the installation.

This unit 3 comprises at least one guard bed and at least one bed of at least one oxidation catalyst, in particular a catalyst for the oxidation of methane. The catalytic removal of the oxygen is carried out in the presence of methane present in the gas stream, in particular in the biomethane or the biogas, and its impurity consisting of oxygen, at a temperature of between 280° C. and 450° C., preferably between 280° C. and 390° C. or between 380° C. and 420° C. The contact time or residence time (Rt) of the gas stream passing through the bed of oxidation catalyst is between 1 s and 2.6 s, at a predetermined volume of catalysts and at a temperature of between 280° C. and 450° C., more particularly between 380° C. and 420° C.

The oxidation of the methane results in the by-products of the reaction, carbon dioxide and water.

After an optional compression in a compressor 2, the gas stream, in particular the biogas or the biomethane, can, before entering the catalytic reaction unit 3, pass through a heat exchanger 17 for heating. On exiting from the deoxygenation catalysis, the deoxygenated gas stream is cooled.

As illustrated, this cooling can be carried out in the abovementioned heat exchanger 17 for heating. That is to say that the streams before and after deoxygenation can be thermally exchanged in the same exchanger 17, in particular countercurrentwise.

Downstream, the installation can also comprise an adsorber 19 configured to remove by-products of the oxidation reaction, carbon dioxide and water, possibly contained in the deoxygenated gas stream. The gas stream exiting from the adsorber 19 subsequently feeds the purification unit 5 or the following unit in the installation, for example the unit included in the purification unit 5, for example the second membrane separation unit, when the catalytic reaction unit 3 is integrated into the purification unit 5, or is conveyed to the pressure control valve 13. The structure and the operation of the catalytic reaction unit 3 which are demonstrated in FIG. 7 can be applied to the catalytic reaction unit of any one of FIG. 1 to FIG. 6.

The present invention is also targeted at a process for the production of deoxygenated biomethane 10 which can be implemented by the installation for the production of deoxygenated biomethane 10 described above.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A process for the production of deoxygenated biomethane (10) having an oxygen concentration below 100 ppm, starting from biogas (1), the process comprising the following steps:
a step of making available biogas (1) containing, in molar proportion, from 30% to 75% of methane, from 15% to 60% of carbon dioxide, and at least one of the following: water, nitrogen, hydrogen sulfide, oxygen, and/or volatile organic compounds (VOCs),
a step of compression of the biogas (1) in a compressor (2),
a step of purification of the biogas in a unit for separation of carbon dioxide from methane (5) to produce biomethane (9), said biomethane (9) comprising, in molar proportion, less than 3% of CO₂ and less than 0.7% of O₂, the step of purification of the biogas comprising a membrane separation treatment comprising at least the following steps:
a step (a) of bringing the biogas into contact with a first membrane separation unit (5a) so as to produce a first permeate (6) enriched in carbon dioxide with respect to the biogas (1) and a first retentate (7) enriched in methane with respect to the biogas (1),
a step (b) of bringing the first retentate (7) into contact with a second membrane separation unit (5b) so as to produce a second permeate (8) enriched in carbon dioxide with respect to the first retentate (7) and a second retentate (9) enriched in methane with respect to the first retentate (7), the second retentate (9) being the biomethane (9), and
a step (c) of bringing the first permeate (6) into contact with a third membrane separation unit (5c) so as to produce a third permeate (11) enriched in carbon dioxide with respect to the first permeate (6) and a third retentate (12) enriched in methane with respect to the first permeate, and
a deoxygenation step in which the first retentate (7) is deoxygenated in at least one catalytic reaction unit (3) comprising at least one bed of at least one methane oxidation catalyst, to produce deoxygenated first retentate (7).

2. The process as claimed in claim 1, comprising:
a step of control of the concentration of carbon dioxide in the deoxygenated biomethane, by regulating the pressure within the purification unit and the catalytic reaction unit, and/or
a step of control of the concentration of oxygen in the deoxygenated biomethane, by regulating the operating temperature within the catalytic reaction unit.

3. The process as claimed in claim 1, comprising a step of pretreatment of the biogas (1), the pretreatment step being configured to remove at least a part of the water and/or of the hydrogen sulfide and/or of the VOCs present in the biogas (1) before the compression step.

4. The process as claimed in claim 1, further comprising, before the deoxygenation step, a step of removal of at least one impurity selected from sulfur compounds, chlorine compounds, halogen compounds and VOCs, by bringing the biogas (1) or the biomethane (9) into contact with at least one impurity removal unit (15), the impurity removal unit (15) comprising at least one guard bed comprising particles of at least one metal oxide of at least one metal chosen from transition metals.

5. The process as claimed in claim 1, in which the step of purification of the biogas comprises a treatment by adsorption in a pressure swing adsorption (PSA) unit and/or a treatment by absorption in a scrubbing column.

6. An installation for the production of deoxygenated biomethane (10) having an oxygen concentration below 100 ppm, starting from biogas (1), the installation comprising:
a unit for the purification (5) of the biogas (1) capable of producing biomethane (9), configured in order to produce biomethane having a molar concentration of less than 5% of CO₂ and less than 1% of O₂, starting from a biogas stream having a concentration of CO₂ above 15% to 60%, the purification unit (5) comprising a unit for treatment by membrane permeation comprising:
a first membrane separation unit (5a) equipped with a first membrane able and configured to receive the biogas and to provide a first permeate (6) and a first retentate (7), said first membrane being more permeable to carbon dioxide than to methane,
a second membrane separation unit (5b) equipped with a second membrane able and configured to receive the first retentate (7) and to provide a second permeate (8) and a second retentate (9), said second membrane being more permeable to carbon dioxide than to methane and said second retentate (9) being the biomethane (9), and
a third membrane separation unit (5c) equipped with a third membrane able and configured to receive the first permeate (6) and to provide a third permeate (11) and a third retentate (12), said third membrane being more permeable to carbon dioxide than to methane,
a compressor (2) configured to compress the biogas (1), and
at least one catalytic reaction unit (3) comprising at least one bed of at least one oxidation catalyst configured to deoxygenate the first retentate (7), the catalytic reaction unit (3) being arranged downstream of the first membrane separation unit (5a) and configured to bring the first retentate (7) into contact with at least one bed of at least one oxidation catalyst of the catalytic reaction unit (3).

7. The installation as claimed in claim 6, comprising:
a device (13) for control of the pressure within the purification unit and/or within the catalytic reaction unit, and/or
a unit for control of the operating temperature within the catalytic reaction unit.

8. The installation as claimed in claim 7, comprising an electronic controller (16) comprising a microprocessor, said controller being configured to regulate the composition of the deoxygenated biomethane (10) produced by operating the device (13) for control of the pressure within the purification unit (5) and/or within the catalytic reaction unit (3), and/or by operating the unit for control of the operating temperature (17, 90, 91) within the catalytic reaction unit (3).

9. The installation as claimed in claim 6, comprising at least one impurity removal unit (15) comprising at least one guard bed, the impurity removal unit (15) being located upstream of the at least one catalytic reaction unit, said guard bed comprising particles of at least one metal oxide of at least one metal chosen from transition metals, said guard bed being placed upstream of said at least one bed of at least one oxidation catalyst.

10. The installation as claimed in claim 6, in which the unit (5) for purification of the biogas (1) comprises a PSA unit and/or a scrubbing column.
